(19) United States
(12) Patent Application Publication
(10) Pub. No.: US 2025/0221905 A1
(43) Pub. Date: Jul. 10, 2025

(54) HAIR CARE COMPOSITION

(71) Applicant: Conopco, Inc., d/b/a UNILEVER, Englewood Cliffs, NJ (US)

(72) Inventors: Jian LIU, Shanghai (CN); Yingying PI, Shanghai (CN); Yisheng YANG, Shanghai (CN)

(73) Assignee: Conopco, Inc., d/b/a UNILEVER, Englewood Cliffs, NJ (US)

(21) Appl. No.: 18/846,707

(22) PCT Filed: Mar. 1, 2023
(86) PCT No.: PCT/EP2023/055063

§ 371 Date: Sep. 13, 2024

(30) Foreign Application Priority Data
Mar. 24, 2022 (WO) PCT/CN2022/082801

(51) Int. Cl. A61K 8/44; A61K 8/34; A61K 8/49; A61K 8/86; A61Q 5/00; A61Q 5/12

(52) U.S. Cl. CPC A61K 8/44; A61K 8/345; A61K 8/4993; A61K 8/86; A61Q 5/008; A61Q 5/12

(57) ABSTRACT

A hair care composition is disclosed comprising i) a non-ionic surfactant comprising fatty alcohol ethoxylates or polyethoxylated monoester wherein having a HLB from 12 to 17; and ii) a sebum control active selected from capryloyl glycine, sarcosine or a mixture thereof; wherein said composition comprises said non-ionic surfactant in an amount from 0.35 to 15% by weight of the composition; wherein said composition comprises at least 40% of water by weight of the composition; wherein the composition is a leave-on composition and viscosity of the composition is not higher than 2000cps.

### FIELD OF THE INVENTION

This invention relates to hair care compositions, preferably leave-on compositions. More particularly the invention relates to hair care compositions that comprise a sebum control active selected from capryloyl glycine, sarcosine or a mixture thereof.

### BACKGROUND OF THE INVENTION

Hair care compositions are formulated for specific purposes, such as cleansing or conditioning benefits or a combination of the two. Preferred hair care composition will provide more benefits than simple cleansing and conditioning.

Scalp or hair becomes soiled due to its contact with the surrounding environment and from sebum secreted from the hair follicles. The build-up of sebum and environmental soiling can cause the hair to have a dirty or greasy feel, and an unattractive appearance. In order to ameliorate these effects, it is necessary to shampoo the hair with regularity. Sebum is naturally present on scalp but is often seen as a negative indicator of beauty and health through association with greasy hair or even a suggested association with more serious conditions such as seborrheic dermatitis.

Sarcosine is a natural occurring amino acid, a methylated derivative of the essential amino acid glycine found in the body. It can also be a precursor of glycine amino acid, and thus considered as taking part to the building of constitutive skin macromolecules like collagen and elastin. In the body, sarcosine comes from diet (egg yolks, legumes) or metabolization of glycine. It has been studied and found its potential to inhibit 5α-reductase 1 gene expression for a decrease in transformation of testosterone in DHT and thus may help to reduce sebum overproduction in the scalp.

Capryloyl glycine is an amide of Glycine (amino acid) and Caprylic acid (a fatty acid that contains eight carbon atoms in the chain). It can offer skin protection & regulation by restoring the “acidic layer”. It has also been found to reduce sebum excess action modeled by 5α-reductase evaluation

The present inventors have now found unexpectedly that the sebum control active selected from capryloyl glycine, sarcosine or a mixture thereof can be stable with a specific non-ionic surfactant in aqueous leave on formulation with high amount of water, thereby deliver desired sensory benefit and sebum control benefit to scalp and/or hair.

US2004096528 A1 (Daniel A.Miser, etc.) discloses a skin creamy cleanser comprising 2 wt % of polysorbate-20, 1 wt % of capryloyl glycine and 63 wt % water.

WO 2013102248 A2 (Johnson&Johnson) discloses a sunscreen composition comprising 0.5 wt % of steareth-21 and polysorbate-20, 0.5 wt % of a mixture comprising capryloyl glycine, sarcosine and 58% water (Examples 2 and 3). It is disclosed in D2 that the viscosity of composition of Example 2 and 3 are higher than 5000 cps.

XP055974989 discloses an anti-dandruff scalp lotion. It comprises polysorbate-20, sarcosine, capryloyl glycine and water. However, it does not disclose the quantity of these ingredients.

XP055974973 discloses an oil control treatment. It comprises oleth-20, capryloyl glycine and water. However, it does not disclose the quantity of these ingredients.

### SUMMARY OF THE INVENTION

In a first aspect, the present invention is directed to hair care composition comprising:
i) a non-ionic surfactant comprising fatty alcohol ethoxylates or polyethoxylated monoester wherein having a HLB from 12 to 17; and
ii) a sebum control active selected from capryloyl glycine, sarcosine or a mixture thereof; wherein said composition comprises said non-ionic surfactant in an amount from 0.35 to 15% by weight of the composition; wherein said composition comprises at least 40% of water by weight of the composition; wherein the composition is a leave-on composition and viscosity of the composition is not higher than 2000 cps.

In a second aspect, present invention is directed to a non-therapeutic method of treating the hair or scalp comprising the step of applying a composition of the first aspect onto the scalp or hair.

In a third aspect, the present invention is directed to a non-therapeutic method of delivering sebum control benefit comprising the step of applying a composition of the first aspect onto hair or scalp surfaces of an individual.

In a fourth aspect, the present invention is directed to a composition of the first aspect for use in delivering sebum control benefit on scalp or hair.

All other aspects of the present invention will more readily become apparent upon considering the detailed description and examples which follow.

### DETAILED DESCRIPTION OF THE INVENTION

### Hair Care Composition

“Hair care composition”, as used herein, is meant to include a composition for topical application to hair and/or scalp of mammals, especially humans. Such a composition may be generally classified as leave-on or rinse off, and includes any product applied to a human body for also improving appearance, cleansing, odor control or general aesthetics. The composition of the present invention can be in the form of a liquid, lotion, cream, foam, scrub, gel, or bar. Non-limiting examples of such compositions include leave-on hair lotions, creams, and rinse-off shampoos, conditioners, shower gels, or toilet bar. The composition of the present invention is preferably hair treatment composition, more preferably a leave-on composition, i.e., a composition intended to be applied to the hair and/or scalp without a rinsing step.

### HLB Value

“HLB value” refers to a value defining the affinity of a surfactant for water or oil. HLB numbers are calculated for non-ionic surfactants, and these surfactants have numbers ranging from 0-20.

### Miscellaneous

Except in the examples, or where otherwise explicitly indicated, all numbers in this description indicating amounts of material or conditions of reaction, physical properties of materials and/or use may optionally be understood as modified by the word “about”.

All amounts are by weight of the final hair care composition, unless otherwise specified. It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### Sebum Control Active

The composition of the present invention comprises a sebum control active selected from capryloyl glycine, sarcosine or a mixture thereof. It is preferred that the composition comprises from 0.01 to 10% of the sebum control active, preferably from 0.05 to 5%, more preferably from 0.05 to 3% by weight of the composition.

Sarcosine according to the present invention is also known as N-methylglycine (C₃H₇NO₂). The CAS number is 107-97-1 and the compound has the general formula (I) as below:

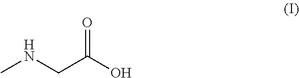

When the sebum control active is sarcosine, the composition may comprise a glycerin.,. Since the inclusion of glycerin will introduce sticky feeling sensory, it is preferred that the composition comprises not higher than 10% of glycerin, more preferably not higher than 5% of glycerin, most preferably not higher than 1% of glycerin by weight of the composition

The composition may comprise from 0.1 to 10% of glycerin, preferably from 0.2 to 5%, furthermore preferably from 0.5 to 2% by weight of the composition, most preferably 0.1 to 1% by weight of the composition.

When the sebum control active is sarcosine, the composition may comprise from 0.01 to 10% of sarcosine, preferably from 0.05 to 5%, more preferably from 0.1 to 3%, furthermore preferably 0.1 to 1% by weight of the composition.

Capryloyl glycine a functional ingredient with many benefits including controlling excessive secretion of sebum. It is also known as N-(1-oxooctyl) glycine (C10H19NO3). The CAS number is 14246-53-8 and the compound has the general formula (II) as below:

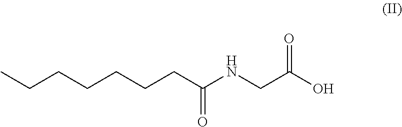

When the sebum control active is capryloyl glycine, the composition may comprise from 0.01 to 10% of capryloyl glycine, preferably from 0.05 to 5%, more preferably from 0.05 to 3%, furthermore preferably from 0.1 to 1.5%, furthermore preferably 0.1 to 1% by weight of the composition. When the sebum control active is capryloyl glycine, to avoid sticky feeling sensory, it is preferred that the composition comprises not higher than 10% of glycerin, more preferably not high than 5% of glycerin, furthermore preferably not higher than 1% of glycerin by weight of the composition, most preferably substantially free from glycerin.

### Non-Ionic Surfactant

The composition of the present invention comprises a non-ionic surfactant comprising fatty alcohol ethoxylates or polyethoxylated monoester wherein having a HLB from 12 to 17, preferably from 15 to 17.

It is preferred that non-ionic surfactant of the present invention comprises C12-C18 fatty alcohol ethoxylates, preferably selected from oleth-10, steareth-10, ceteth-10, ceteareth-12, laureth-9, steareth-20, oleth-20, ceteth-20, ceteareth-20, ceteareth-25, c12-13 pareth-23, laureth-23, more preferably selected from steareth-20, oleth-20, ceteth-20, ceteareth-20, furthermore preferably selected from oleth-20 and steareth-20, most preferably is oleth-20.

It is preferred that non-ionic surfactant of the present invention comprises polyethoxylated monoester, preferably selected from polysorbate 21, polysorbate 60, polysorbate 80, polysorbate 40, polysorbate 20, more preferably comprises polysorbate 20.

The composition of the present invention comprises the non-ionic surfactant in an amount from 0.35 to 15% by weight of the composition, preferably from 0.4 to 10%, more preferably from 0.5 to 5%, furthermore preferably from 0.5 to 3% by weight of the composition.

Typically leave-on compositions are substantially free from cleansing surfactants. Thus it is preferred that the composition comprises less than 1 wt % cleansing surfactant, more preferably less than 0.5 wt %, more preferably still less than 0.1% and most preferably from 0 to 0.01 wt %. Cleansing surfactants are typically anionic surfactants such as, for example, alkyl sulfates and/or alkyl ether sulfates.

For the desired sensory benefit, it is preferred that the composition of the present invention does not comprises other surfactant in addition to the non-ionic surfactant above.

### Moisturizing Agent

It is preferred that compositions of the invention comprise a moisturizing agent. Preferably the moisturizing agent is selected from the group consisting of 1,2-glycols, selected from caprylyl glycol, pentylene glycol, 1,2-hexanediol and C15-18 glycol.

The moisturizing agent can also be hyaluronic acid.

The composition of the invention may comprise 0.01 to 10% of the moisturizing agent, preferably 0.1 to 5%, more preferably 0.3 to 2% by weight of the composition.

### Hair Care Composition

The hair care composition in accordance with the present invention comprises an aqueous cosmetically acceptable carrier. In order to deliver desired sensory (ex. watery sensory, clean and fresh), the composition of the present invention comprises at least 40% of water, preferably from 40 to 98% of water, more preferably from 60% to 98% of water, evenmore preferably from 65% to 95% of water, furthmore preferably from 70% to 95% furthermore preferably from 80% to 95% of water by weight of the composition.

“Lipophilic material” for the purpose of the present invention means a material which tends to combine with or dissolve in lipids, oils or fats. In order to deliver the desired sensory, the composition of the present invention may comprise not high than 10% of lipophilic material, preferably not high than 5% of lipophilic material, more preferably not higher than 2% of lipophilic material, furthermore preferably not higher than 1% of lipophilic material, evenmore preferably not higher than 0.5% of lipophilic material by weight of the composition. Lipophilic material may comprise an oil, a fatty alcohol, a fatty acid, a fatty ester, a wax, a silicone, etc. The hair care composition in accordance with the present invention is a leave-on composition, ex., a hair lotion, hair serum.

In order to deliver the desired sensory (ex., clean and fresh), the composition in accordance with the present invention has viscosity not higher than 2000 cps, preferably not higher than 1000 cps, more preferably not higher than 100 cps. It is preferred that the viscosity of the composition is from 1 to 2000 cps, more preferably from 2 to 1500 cps, furthermore preferably from 5 to 1000 cps, most preferably from 10 to 100 cps.

It is preferred that the composition is a liquid solution, and it can be a hair lotion or hair serum. It is more preferred that the composition is a transparent solution.

It is preferred that the transmittance of the composition is at least 85%, preferably at least 90%, more preferably at least 95%. The transmittance of the composition can be measured at wavelength of 400 nm.

It is preferred that the pH of the composition is from 4 to 6, more preferably from 4.5 to 5.5.

Preferably, the composition may comprise an antidandruff agent. Antidandruff agents are compounds that are active against dandruff and are typically antimicrobial agents, preferably antifungal agents. Antidandruff agents typically display a minimum inhibitory concentration of about 50 mg/ml or less against Malassezia.

Whenever present, the hair care composition of the invention preferably comprises 0.01 to 5 wt %, more preferably 0.1 to 2%, furthermore preferably 0.5 to 1% of the antidandruff agent by weight of the composition.

The antidandruff agent is preferably selected from piroctone compound, azoles, salicylic acid and combinations thereof. Azoles include ketoconazole and climbazole, preferably climbazole. It is preferred that the antidandruff agent is piroctone compound, more preferably piroctone olamine.

The hair care composition may additionally comprise a conditioning agent to provide conditioning benefit. Preferably, the hair care composition comprises discrete dispersed droplets of a water-insoluble conditioning agent, which has a mean droplet diameter (D3,2) of less than 15 microns, preferably less than 10 microns, more preferably less than 5 microns, most preferably less than 3 microns. The mean droplet diameter (D3,2) of a water-insoluble conditioning agent may be measured by means of a laser light scattering technique, for example using a 2600D Particle Sizer from Malvern Instruments.

The water-insoluble conditioning agent may include non-silicone conditioning agent comprising non-silicone oily or fatty materials such as hydrocarbon oils, fatty esters and mixtures thereof. Preferably, the water-insoluble conditioning agent is emulsified silicone oil.

Suitable silicones include polydiorganosiloxanes, in particular polydimethylsiloxanes which have the CTFA designation dimethicone. Also suitable for use in compositions of this invention (particularly shampoos and conditioners) are polydimethyl siloxanes having hydroxyl end groups, which have the CTFA designation dimethiconol. Also suitable for use in compositions of this invention are silicone gums having a slight degree of cross-linking, as are described for example in WO 96/31188. Preferably, the silicone oil comprises dimethicone, dimethiconol or a mixture thereof.

Suitable emulsified silicones for use in the hair care compositions of this invention are available as pre-formed silicone emulsions from suppliers of silicones such as Dow Corning and GE silicones. The use of such pre-formed silicone emulsion is preferred for ease of processing and control of silicone particle size. Such pre-formed silicone emulsions will typically additionally comprise a suitable emulsifier, and may be prepared by a chemical emulsification process such as emulsion polymerisation, or by mechanical emulsification using a high shear mixer. Examples of suitable pre-formed silicone emulsions include DC1785, DC1788, DC7128, all available from Dow Corning. These are emulsions of dimethiconol/dimethicone.

Another class of silicones which may be used are functionalized silicones such as amino functional silicones, meaning a silicone containing at least one primary, secondary or tertiary amine group, or a quaternary ammonium group. Examples of suitable amino functional silicones include polysiloxanes having the CTFA designation “amodimethicone.”

The water-insoluble conditioning agent is generally present in hair care composition of this invention in an amount from 0.05 to 15%, preferably from 0.1 to 10%, more preferably from 0.5 to 8%, most preferably from 1 to 5%, based on total weight of the hair care composition.

Preservatives may also be incorporated into the hair care composition of this invention to protect against the growth of potentially harmful microorganisms. Suitable traditional preservatives include alkyl esters of parahydroxybenzoic acid, hydantoin derivatives, propionate salts, and a variety of quaternary ammonium compounds. Illustrative yet non-limiting examples of the types of preservatives that may be used in this invention include, for examples, phenoxyethanol, sodium salicylate, methyl paraben, butyl paraben, propyl paraben, diazolidinyl urea, sodium dehydroacetate, benzyl alcohol, sodium benzoate, iodopropynyl butylcarbamate, caprylyl glycol, disodium EDTA or mixtures thereof. In an especially preferred embodiment, the preservative is sodium benzoate, phenoxyethanol, sodium salicylate or a mixture thereof. Preservatives are preferably employed in amounts ranging from 0.01 to 2% by weight of the hair care composition.

It is further preferred that the hair care composition of the invention comprises a cosmetic ingredient. Preferably the cosmetic ingredient is selected from the group consisting of a silicone, an antibacterial agent other than antidandruff agents, a foam booster, a perfume, encapsulates (for example encapsulated fragrance) a dye, a colouring agent, a pigment, a preservative, a thickener, a protein, a phosphate ester, a buffering agent, a pH adjusting agent, a pearlescer (for example; mica, titanium dioxide, titanium dioxide coated mica, ethylene glycol distearate (INCI glycol distearate)) and/or opacifier, a viscosity modifier, an emollient, a sunscreen, an emulsifier, a sensate active (for example menthol and menthol derivatives), vitamins, mineral oils, essential oils, lipids, natural actives, glycerin, natural hair nutrients such as botanical extracts, fruit extracts, sugar derivatives and amino acids, microcrystalline cellulose and mixtures thereof.

Preferably, the composition of the present invention includes from 0.01 to 20 wt % of the at least one cosmetic ingredient, more preferably from 0.05 to 10 wt %, still more preferably from 0.075 to 7.5 wt % and most preferably, from 0.1 to 5 wt % of the at least one cosmetic ingredient, by weight of the total composition.

The composition of the present invention may also comprise synergistic antimicrobial compounds that give synergistic antimicrobial benefit when used in combination with the antimicrobial active to enhance its properties and further inhibit the growth of bacterial/fungal. Non-limiting examples of these compounds include compounds having alcoholic groups (e.g. honokiol, magnolol or paeonol), Piperazines and a phenolic compound found in natural plant extract viz. thymol and terpeniol.

### Method and Use

The present invention provides for a method of treating the scalp or hair comprising application of a composition of the first aspect onto scalp or hair. The method is non-therapeutic in nature. Preferably it is for cosmetic purpose.

The present invention provides for a method of delivering sebum control benefit comprising the step of applying a composition of the first aspect onto hair or scalp surfaces of an individual. The method is non-therapeutic in nature. Preferably it is for cosmetic purpose.

The present invention also provides for use of the composition of the first aspect for use in delivering sebum control benefit on scalp or hair. The use is non-therapeutic in nature. Preferably it is for cosmetic purpose. It also provides for use of the composition of the first aspect for use in delivering sensory benefit including clean and fresh sensory.

### Mode of Use

The compositions of the invention are primarily intended for topical application to scalp and/or at least a portion of the hair of an individual, either in rinse-off or leave-on compositions, preferably in leave-on compositions, i.e., lotions or serums.

When the composition is a leave-on composition, it is topically applied onto scalp directly and/or to the hair and then massaged into the hair and scalp.

In order to allow maximum opportunity for the composition to contact the hair and/or scalp and deliver sebum-control benefit, it is preferred that the leave-on composition is preferably left on the hair/scalp for at least one hour, more preferably from 2 to 24 hours before rinsing and/or washing the hair/scalp.

The invention will be further illustrated by the following, non-limiting Examples, in which all percentages quoted are by weight based on total weight unless otherwise stated.

The examples are intended to illustrate the invention and are not intended to limit the invention to those examples per se.

### EXAMPLES

Non-ionic surfactants listed in Table 1 were put through some tests as detailed hereinafter.

TABLE 1

| Trade name | INCI name | HLB value | Supplier |
|---|---|---|---|
| Brij S2 | Steareth-2 | 4.9 | Croda |
| Tween 85 | Polysorbate 85 | 11 | Croda |
| PEG-100 Stearate | Glyceryl Stearate | 18.8 | Croda |
| Brij S20 | Steareth-20 | 15.3 | Croda |
| Tween 20 | Polysorbate 20 | 16.7 | Croda |
| Brij O20 | Oleth-20 | 15.5 | Croda |

### Example 1

This example demonstrated the non-ionic surfactant within the scope of the present invention can improve the stability of the composition comprising the sebum control active which is sarcosine in accordance with the present invention. The compositions according to the formulations detailed in Table 2 are prepared. All ingredients are expressed by weight percent of the total formulation, and as level of active ingredient.

TABLE 2

| | Samples | | | | | |
|---|---|---|---|---|---|---|
| Ingredient | A | B | C | 1 | 2 | 3 |
| Water | To 100 | To 100 | To 100 | To 100 | To 100 | To 100 |
| Sarcosine | 0.07 | 0.07 | 0.07 | 0.07 | 0.07 | 0.07 |
| Steareth-2 | 3.00 | — | — | — | — | — |
| Polysorbate 85 | — | 3.00 | — | — | — | — |
| Glyceryl Stearate | — | — | 3.00 | — | — | — |
| Steareth-20 | — | — | — | 3.00 | — | — |
| Polysorbate 20 | — | — | — | — | 3.00 | |
| Oleth-20 | — | — | — | — | — | 3.00 |
| Preservative | 1.1 | 1.1 | 1.1 | 1.1 | 1.1 | 1.1 |
| Perfume | 0.20 | 0.20 | 0.20 | 0.20 | 0.20 | 0.20 |
| PH adjuster | Adjust to | Adjust to | Adjust to | Adjust to | Adjust to | Adjust to |
| | pH 4.5-5.5 | pH 4.5-5.5 | pH 4.5-5.5 | pH 4.5-5.5 | pH 4.5-5.5 | pH 4.5-5.5 |

### Methods

The appearance of the compositions is observed after 24 hours. If the composition is well emulsified and stable, it is clear with no phase separation or visible solids sedimentation.

Unstable formulations are defined as there is phase separation or turbid. The viscosity of the compositions was tested at room temperature. The transmittance of the compositions was tested by using an equipment which is Cary Series UV-Vis Spectrophotometer. The transmittance values of the compositions were measured at wavelength of 400 nm.

### Results

The appearance of the compositions is summarized in Table 3.

TABLE 3

| Samples | A | B | C | 1 | 2 | 3 |
|---|---|---|---|---|---|---|
| Appearance | Turbid | Turbid | Turbid | Clear | Clear | Clear |
| Viscosity (cps) | — | — | — | 40 | 20 | 40 |
| Transmittance (%) | — | — | — | 93.0 | 95.2 | 96.6 |

Samples 1 to 3 comprising combination of specific non-ionic surfactant and sebum active within the scope of the present invention showed good stability as compared to samples A-C which are outside the scope of the present invention.

### Example 2

This example demonstrated the non-ionic surfactant within the scope of the present invention can improve the stability of the composition comprising the sebum control active which is capryloyl glycine in accordance with the present invention. The compositions according to the formulations detailed in Table 4 are prepared. All ingredients are expressed by weight percent of the total formulation, and as level of active ingredient.

TABLE 4

| | Samples | | | | | |
|---|---|---|---|---|---|---|
| Ingredient | D | E | F | 4 | 5 | 6 |
| Water | To 100 | To 100 | To 100 | To 100 | To 100 | To 100 |
| Capryloyl Glycine | 1.00 | 1.00 | 1.00 | 1.00 | 1.00 | 1.00 |
| Steareth-2 | 3.00 | — | — | — | — | — |
| Polysorbate 85 | — | 3.00 | — | — | — | — |
| Glyceryl Stearate | — | — | 3.00 | — | — | — |
| Steareth-20 | — | — | — | 3.00 | — | — |
| Polysorbate 20 | — | — | — | — | 3.00 | |
| Oleth-20 | — | — | — | — | — | 3.00 |
| Preservative | 1.1 | 1.1 | 1.1 | 1.1 | 1.1 | 1.1 |
| Perfume | 0.20 | 0.20 | 0.20 | 0.20 | 0.20 | 0.20 |
| PH adjuster | Adjust to | Adjust to | Adjust to | Adjust to | Adjust to | Adjust to |
| | pH 4.5-5.5 | pH 4.5-5.5 | pH 4.5-5.5 | pH 4.5-5.5 | pH 4.5-5.5 | pH 4.5-5.5 |

### Methods

The appearance of the compositions is observed after 24 hours, the viscosity and transmittance of the compositions are tested using the method as disclosed in Example 1.

### Results

The appearance of the compositions is summarized in Table 5.

TABLE 5

| Samples | D | E | F | 4 | 5 | 6 |
|---|---|---|---|---|---|---|
| Appearance | Turbid | Turbid | Turbid | Clear | Clear | Clear |
| Viscosity (cps) | — | — | — | 40 | 20 | 40 |
| Transmittance(%) | — | — | — | 89.3 | 95.9 | 97.0 |

Samples 4 to 6 comprising combination of specific non-ionic surfactant and sebum active within the scope of the present invention showed good stability as compared to samples D-F which are outside the scope of the present invention.

### Example 3

This example demonstrated the impact of dosage of non-ionic surfactant according to the present invention on the stability of the compositions comprising sebum control active which is sarcosine. The compositions according to the formulations detailed in Table 6 are prepared. All ingredients are expressed by weight percent of the total formulation, and as level of active ingredient.

TABLE 6

| | Samples | | | | | |
|---|---|---|---|---|---|---|
| Ingredient | G | H | 7 | 8 | 9 | 10 |
| Water | To 100 | To 100 | To 100 | To 100 | To 100 | To 100 |
| Sarcosine | 0.07 | — | 0.07 | 0.07 | — | — |
| Capryloyl Glycine | — | 1.00 | — | — | 1.00 | 1.00 |
| Oleth-20 | 0.30 | 0.30 | 0.50 | 1.00 | 0.50 | 1.00 |
| Preservative | 1.1 | 1.1 | 1.1 | 1.1 | 1.1 | 1.1 |
| Perfume | 0.20 | 0.20 | 0.20 | 0.20 | 0.20 | 0.20 |
| PH adjuster | Adjust to | Adjust to | Adjust to | Adjust to | Adjust to | Adjust to |
| | pH 4.5-5.5 | pH 4.5-5.5 | pH 4.5-5.5 | pH 4.5-5.5 | pH 4.5-5.5 | pH 4.5-5.5 |

### Methods

The appearance of the compositions is observed after 24 hours, the viscosity and transmittance of the compositions are tested using the method as disclosed in Example 1.

### Results

The appearance of the compositions is summarized in Table 7.

TABLE 7

| Samples | G | H | 7 | 8 | 9 | 10 |
|---|---|---|---|---|---|---|
| Appearance | Turbid | Turbid | Clear | Clear | Clear | Clear |
| Viscosity (cps) | — | — | 50 | 40 | 40 | 50 |
| Transmittance (%) | — | — | 96.9 | 97.4 | 96.8 | 96.2 |

Samples 7 to 10 comprising combination of specific non-ionic surfactant and sebum active having the dosage of non-ionic surfactant within the scope of the present invention showed good stability as compared to samples G-H which are outside the scope of the present invention.

### Example 4

This example demonstrated the composition of the invention can deliver sebum control sensory to hair or scalp. The leave-on compositions according to the formulations detailed in Table 8 are prepared. All ingredients are expressed by weight percent of the total formulation, and as level of active ingredient.

TABLE 8

Samples

| Ingredient | I | 11 |
|---|---|---|
| Water | To 100 | To 100 |
| Oleth-20 | 3 | 3 |
| Sebum control active | — | 1.00 |
| Preservative | 0.70 | 0.70 |
| Perfume | 0.20 | 0.20 |
| PH adjuster | Adjust to | Adjust to |
| | pH 4.5-5.5 | pH 4.5-5.5 |

The viscosity and transmittance of sample 11 is tested using the method as disclosed in Example 1. The viscosity is 40 cps and the transmittance is about 97.0%.

The compositions (Sample 11 vs. Sample I) in Table 8 were used by 90 consumers (greasy scalp) and scored.

The results showed that Sample 11 was significantly (at least 95% conf.) higher in the following attributes comparing with Sample I (placebo):
Obvious oil control efficacy
Obviously feel scalp greasy condition improved
Hair or scalp is fresh and not greasy after use

It can be concluded from the results that the stable formulation Sample 11 within the scope of the present invention can deliver the sebum control benefit especially removing greasiness for hair or scalp.

1. A hair care composition comprising:
i) a non-ionic surfactant comprising fatty alcohol ethoxylates or polyethoxylated monoester wherein the non-ionic surfactant has a HLB from 12 to 17; and
ii) a sebum control active selected from capryloyl glycine, sarcosine, or a mixture thereof;
wherein the composition comprises the non-ionic surfactant in an amount from 0.35 to 15% by weight of the composition;
wherein the composition comprises at least 40% of water by weight of the composition; and
wherein the composition is a leave-on composition and viscosity of the composition is not higher than 2000 cps.

2. The composition as claimed in claim 1, wherein the non-ionic surfactant comprises C12-C18 fatty alcohol ethoxylates.

3. The composition as claimed in claim 1, wherein the non-ionic surfactant comprises the polyethoxylated monoester, selected from polysorbate 21, polysorbate 60, polysorbate 80, polysorbate 40, or polysorbate 20.

4. The composition as claimed in claim 1, wherein the composition comprises the non-ionic surfactant in an amount of from 0.4 to 10%, by weight of the composition.

5. The composition as claimed in claim 1, wherein the composition comprises the sebum control active in amount of from 0.01 to 10%, preferably from 0.05 to 5% by weight of the composition.

6. The composition as claimed in claim 1, wherein the composition comprises water in amount of from 40 to 98%, by weight of the composition.

7. The composition as claimed in claim 1, wherein the composition comprises glycerin when the sebum control active is sarcosine.

8. The composition as claimed in claim 1, wherein the composition comprises a moisturizing agent.

9. The composition as claimed in claim 1, wherein a pH of the composition is from 4 to 6, preferably from 4.5 to 5.5.

10. The composition as claimed in claim 1, wherein the composition does not comprise other surfactants in addition to the non-ionic surfactant.

11. The composition as claimed in claim 1, wherein the composition comprises net high-no higher than 10% of a lipophilic material.

12. The composition as claimed in claim 1, wherein the composition is a liquid solution.

13. A method of treating the scalp or hair comprising applying a composition as claimed in claim 1 to the scalp or hair.

14. A method of delivering sebum control benefit comprising applying a composition as claimed in claim 1 onto hair or scalp surfaces of an individual.

15. (canceled)

16. The composition as claimed in claim 2, wherein the C12-C18 fatty alcohol ethoxylates are selected from oleth-10, steareth-10, ceteth-10, ceteareth-12, laureth-9, steareth-20, oleth-20, ceteth-20, ceteareth-20, ceteareth-25, C12-13 pareth-23, or laureth-23.

17. The composition as claimed in claim 4, wherein the composition comprises the non-ionic surfactant in an amount of from 0.5 to 5%, by weight of the composition.

18. The composition as claimed in claim 4, wherein the composition comprises the non-ionic surfactant in an amount of from 0.5 to 3%, by weight of the composition.

19. The composition as claimed in claim 5, wherein the composition comprises the sebum control active in amount of from 0.05 to 5%, by weight of the composition.

20. The composition as claimed in claim 6, wherein the composition comprises water in amount of from 80 to 95%, by weight of the composition.

21. The composition as claimed in claim 9, wherein the pH of the composition is from 4.5 to 5.5.
